(19) United States
(12) Patent Application Publication
(10) Pub. No.: US 2010/0215699 A1
(43) Pub. Date: Aug. 26, 2010

(54) LACTOFERRIN AS A RADIOPROTECTIVE AGENT

(75) Inventors: Atul Varadhachary, Houston, TX (US); Karel Petrak, Houston, TX (US); Paul Blezinger, Houston, TX (US)

Correspondence Address: FULBRIGHT & JAWORSKI, LLP, 1301 MCKINNEY, SUITE 5100, HOUSTON, TX, 77010-3095, US

(73) Assignee: AGENNIX INCORPORATED, Houston, TX (US)

(21) Appl. No.: 12/305,944

(22) PCT Filed: Jun. 22, 2007
(86) PCT No.: PCT/US07/71942

§ 371 Date: Feb. 26, 2009

Related U.S. Application Data
(60) Provisional application No. 60/905,540, filed on Mar. 7, 2007.

(51) Int. Cl. A61K 38/40; A61K 9/00; A61K 38/19; A61P 31/00; A61P 31/10; A61P 31/12; A61P 31/04; A61P 39/00

(52) U.S. Cl. 424/400; 514/8; 424/85.1

(57) ABSTRACT

This present invention relates to the field of protecting against, or rectifying the effects of damaging ionizing irradiation. The method of treatment involves oral administration of a lactoferrin composition, alone or in combination with other treatments, both in combination with other radio-protective agents and/or the standard of care. Further, the method of treatment provides for a topical administration of lactoferrin to treat lesions caused by local damaging irradiation.

### TECHNICAL FIELD

This invention relates to the field of medicine, more specifically, to the use of lactoferrin as a radioprotective agent. Lactoferrin is used to protecting against, or rectifying the effects of damaging ionizing irradiation and increasing survival of animals.

### BACKGROUND OF THE INVENTION

Ionizing radiation has an adverse effect on cells and tissues, primarily through cytotoxic effects. In humans, exposure to ionizing radiation occurs primarily through therapeutic techniques (such as anticancer radiotherapy) or through occupational and environmental exposure.

A major source of exposure to ionizing radiation is the administration of therapeutic radiation in the treatment of cancer or other proliferative disorders. Subjects exposed to therapeutic doses of ionizing radiation typically receive between 0.1 and 2 Gy per treatment, and can receive as high as 5 Gy per treatment. Depending on the course of treatment prescribed by the treating physician, multiple doses may be received by a subject over the course of several weeks to several months.

Occupational doses of ionizing radiation may be received by persons whose job involves exposure (or potential exposure) to radiation, for example in the nuclear power and nuclear weapons industries. There are currently 104 nuclear power plants licensed for commercial operation in the United States. Internationally, a total of 430 nuclear power plants are operating in 32 countries. All personnel employed in these nuclear power plants may be exposed to ionizing radiation in the course of their assigned duties. Incidents such as the Mar. 28, 1979 accident at Three Mile Island nuclear power plant, which released radioactive material into the reactor containment building and surrounding environment, illustrate the potential for harmful exposure. Even in the absence of catastrophic events, workers in the nuclear power industry are subject to higher levels of radiation than the general public.

Military personnel stationed on vessels powered by nuclear reactors, or soldiers required to operate in areas contaminated by radioactive fallout, risk similar exposure to ionizing radiation. Occupational exposure may also occur in rescue and emergency personnel called in to deal with catastrophic events involving a nuclear reactor or radioactive material. For example, the men who fought the Apr. 26, 1986 reactor fire at the Chernobyl nuclear power plant suffered radiation exposure, and many died from the radiation effects. In August 2000, navy and civilian rescue personnel risked exposure to radiation when attempting to rescue the crew of the downed Russian nuclear-powered submarine Kursk. Salvage crews may still face radiation exposure if the submarine's reactor plant was damaged.

Other sources of occupational exposure may be from machine parts, plastics, and solvents left over from the manufacture of radioactive medical products, smoke alarms, emergency signs, and other consumer goods. Occupational exposure may also occur in persons who serve on nuclear powered vessels, particularly those who tend the nuclear reactors, in military personnel operating in areas contaminated by nuclear weapons fallout, and in emergency personnel who deal with nuclear accidents.

Humans and other animals (such as livestock) may also be exposed to ionizing radiation from the environment. The primary source of exposure to significant amounts of environmental radiation is from nuclear power plant accidents, such as those at Three Mile Island, Chernobyl and Tokaimura. A 1982 study by Sandia National Laboratories estimated that a “worst-case” nuclear accident could result in a death toll of more than 100,000 and long-term radioactive contamination of large areas of land.

For example, the estimated number of deaths from the Chernobyl accident is from 8,000 to 300,000, and in the Ukraine alone, over 4.6 million hectares of land was contaminated with varying levels of radiation. Fallout was detected as far away as Ireland, northern Scandinavia, and coastal Alaska in the first weeks after the accident. 135,000 people were evacuated from a 30-mile radius “dead zone” around the Chernobyl plant, an area which is still not fit for human habitation. Approximately 1.2 million people continue to live in areas of low-level radiation outside the “dead-zone.”

Other nuclear power plant accidents have released significant amounts of radiation into the environment. The Three Mile Island accident was discussed above. In Japan, a cracked pipe leaked 51 tons of coolant water from the Tsuruga 2 nuclear plant in July of 1999. A more serious accident occurred on Sep. 30, 1999 at a uranium reprocessing facility in Tokaimura, Japan, where 69 people received significant radiation exposure. The accident occurred when workers inadvertently started a self-sustaining nuclear chain reaction, causing a release of radiation into the atmosphere. A radiation count of 0.84 mSv/hour (4000 times the annual limit) was detected in the immediate area. Thirty-nine households (150 people) were evacuated and 200 meter radius around the site was declared off-limits. The roads within a 3 kilometer radius of the site were closed and residents within 10 kilometer radius of the site were advised to stay indoors. The Tokaimura “criticality event” is ranked as the third most serious accident—behind Three Mile Island and Chernobyl—in the history of the nuclear power industry.

Environmental exposure to ionizing radiation may also result from nuclear weapons detonations (either experimental or during wartime), discharges of actinides from nuclear waste storage and processing and reprocessing of nuclear fuel, and from naturally occurring radioactive materials such as radon gas or uranium. There is also increasing concern that the use of ordnance containing depleted uranium results in low-level radioactive contamination of combat areas.

Delayed, irreversible changes of the skin, radiation dermatitis or Radiodermatitis, usually do not develop as a result of sublethal whole-body irradiation, but instead follow higher doses limited to the skin. These changes could occur, for example, if there is heavy contamination of bare skin with beta-emitting materials. Table 4 lists the degrees of radiation dermatitis for local skin area radiation doses.

TABLE 4
Radiation dermatitis.

| Radiation | Dose | Effect |
|---|---|---|
| Acute | 6-20 Sv | Erythema only |
| | 20-40 Sv | Skin breakdown in 2 wk |
| | >3000 Sv | Immediate skin blistering |
| Chronic | >20 Sv | Dermatitis, with cancer risk |

Radiation-induced damage may be repairable, but in some cases the repair is inaccurate, resulting in adverse health effects within a short time of hours to weeks or delayed effects observable many months or years after exposure. Radiation-induced mutations in a germ cell can lead to heritable changes that may not be expressed for many generations. The manifestation of adverse health effects, of course, depends on the radiation dose, duration of exposure, differentiation and sensitivity of the tissues, and intrinsic antioxidant defense mechanism(s).

Ionizing radiation is capable of depleting or suppressing the immune system. Much of the suppression can be attributed to cell damage or death caused directly by irradiation or by cell death or malfunction due to protein damage, DNA or RNA strand breakage, by inhibition of DNA synthesis, etc. There is a pressing need to identify non-toxic agents for prophylaxis and recovery from radiation damage, to be used by personnel at risk of exposure and for the treatment of those exposed to damaging ionizing irradiation.

Acute effects of high-dose radiation include hematopoietic cell loss, immune suppression, mucosal (gastrointestinal and oral) damage, and potential injury to other sites such as the lung, kidney, and central nervous system. Long-term effects, as a result of both high- and low-dose radiation, include dysfunction or fibrosis in a wide range of organs and tissues, and cancer. These changes reflect on the quality of life and mortality of a population.

Infection is the primary cause of death from doses of ionizing radiation that induce hematopoietic and GI syndromes. High-dose radiation with accompanying GI damage results in bacterial translocation from the intestine to other sites in the body and increases mortality.

Pharmaceutical radioprotectants offer a cost-efficient, effective and easily available alternative to radioprotective gear. However, previous attempts at radioprotection of normal cells with pharmaceutical compositions have not been entirely successful. For example, cytokines directed at mobilizing the peripheral blood progenitor cells confer a myeloprotective effect when given prior to radiation (Neta et al., Semin. Radiat. Oncol. 6:306-320, 1996), but do not confer systemic protection. Other chemical radioprotectors administered alone or in combination with biologic response modifiers have shown minor protective effects in mice, but application of these compounds to large mammals was less successful, and it was questioned whether chemical radioprotection was of any value (Maisin, J. R., Bacq and Alexander Award Lecture. “Chemical radioprotection: past, present, and future prospects”, Int J. Radiat Biol. 73:443-50, 1998). Pharmaceutical radiation sensitizers, which are known to preferentially enhance the effects of radiation in cancerous tissues, are clearly unsuited for the general systemic protection of normal tissues from exposure to ionizing radiation.

Because radiation-induced cellular damage is attributed primarily to the harmful effects of free radicals, molecules with direct free radical scavenging properties are particularly promising as radioprotectors. The best-known radioprotectors are the sulfhydryl compounds, such as cysteine and cysteamine. However, these compounds produce serious side effects, such as nausea and vomiting, and are considered to be toxic at the doses required for radioprotection. Amifostine (WR-2721), although approved by the Food and Drug Administration for use in radiotherapy clinics, and also reportedly carried by U.S. astronauts on lunar trips in the event of a solar flare, has a side effects profile that makes it unsuitable for emergency personnel who must engage in demanding rescue and evacuation activities. The side effects include hypotension, nausea, vomiting, sneezing, hot flashes, mild somnolence, and hypocalcemia, and are severe enough to limit the amount of the drug required to levels lower than necessary to achieve maximal radioprotection. Furthermore, amifostine is effective only when administered intravenously (i.v.) or subcutaneously (s.c.), and hence its practical administration is difficult and its utility in open-field terrorism is especially low. Another radio-protective agent, Cystapos (WR-638) is effective only when administered i.v. Another compound, d-CON (WR-1607), or rat poison (which kills by cardiac arrest), seems to be much more effective than amifostine and is capable of producing an equivalent protection at 1/100th of the dose. However, similar to amifostine, d-CON was found to be unusable because of its extreme toxicity. Another agent, androstenediol, which boosts the hematopoietic system, although it has been proposed as a prophylactic drug against ionizing radiation, it has so far been evaluated only in experimental animals. Potassium iodide (IOSAT™ KI) is the only Food and Drug Administration-approved, foil-sealed thyroid blocking drug for preventing thyroid cancer in people exposed to radioactive iodine during radiation emergencies. This drug has been suggested for use not only in the 10-mile emergency planning zone but also in any or all areas potentially affected. KI saturates the thyroid gland with stable iodine and thus prevents the absorption of radioactive iodine by the thyroid gland. However, radioactive iodine, which the KI protects against, is a byproduct of nuclear fission, which takes place only within nuclear reactors (as it did during the Chernobyl disaster) and may not be present during detonation of a “dirty bomb”, limiting KI's utility.

Although the ability of most of the known radioprotectors against the damage caused by ionizing radiation with low linear energy transfer (expressed as KeV/μm) such as gamma rays and X-rays (from 0.2 to 2.0 KeV/μm) is documented, their effectiveness against the damage induced by high linear energy transfer radiation such as protons, neutrons, and alpha particles (from 4.7 to >150 KeV/μm), as occurs in the detonation of nuclear devices, has yet to be thoroughly investigated.

The potential utility of melatonin as a protector against ionizing radiation is worth mentioning here. The hydroxyl and other free radical scavenging efficiency of melatonin, along with its indirect antioxidant properties, have been repeatedly documented in numerous independent investigations (over 900 publications in the literature). Animals subjected to whole-body irradiation and given melatonin exhibited increased survival (LD_{50/30} as well as lethal radiation dose); the protection against radiation-induced oxidative damage is apparent in not only hematopoietic but also other tissues. More importantly, unlike amifostine, melatonin administered orally results in higher circulating levels and more rapidly increasing tissue concentrations.

Thus, there is still an urgent need to identify novel, nontoxic, effective, and convenient compounds to protect humans from the damaging effects of ionizing radiation. The present invention is the first to use oral lactoferrin composition as prophylaxis or treatment of damage to the body inflicted by ionizing radiation and improving patient survival.

### BRIEF SUMMARY OF THE INVENTION

The present invention is directed to a method of treating prophylactically or therapeutically body damage resulting from exposure to ionizing radiation and improving patient survival. The method of treatment involves oral administration of a lactoferrin composition, alone or in combination with other treatments (for example, other radioprotective agents). In another embodiment, lactoferrin presented in a topical formulation is used to treat skin lesions resulting from a localized damaging irradiation.

The following numbered sentences more readily define the invention as described herein.
1. A method of treating a subject exposed to irradiation comprising the step of administering to the subject an effective amount of a lactoferrin composition, wherein said lactoferrin composition decreases morbidity and/or mortality of the subject exposed to irradiation.
2. The method of sentence 1 when said lactoferrin composition is administered prior to exposure to irradiation.
3. The method of sentence 1 when said lactoferrin composition is administered after the exposure to irradiation.
4. The method of sentence 1, wherein said lactoferrin composition is dispersed in a pharmaceutically acceptable carrier.
5. The method of sentence 1, wherein the amount of the lactoferrin composition that is administered is about 0.01 to 2.0 g/kg per day.
6. The method of sentence 1, wherein the amount of the lactoferrin composition that is administered is from 0.01 to 0.5 g/kg.
7. The method of sentence 1, wherein the lactoferrin composition is administered orally.
8. The method of sentence 7, wherein the said lactoferrin composition is administered as a liquid formulation.
9. The method of sentence 7, wherein the said lactoferrin composition is administered as a solid formulation.
10. The method of sentence 9, wherein the said solid formulation comprises an enteric coating.
11. The method of sentence 1, wherein the lactoferrin composition is administered topically.
12. The method of sentence 1, wherein the irradiation is selected from ²³⁵U, ¹³¹I, ¹²³I, ⁹⁹Tc, ²⁰¹Th, ¹³³Xe, ¹²⁵I, ⁶⁰Co, and ¹³⁷Cs, ⁶⁰Co, ¹³⁷Cs, ¹⁹²Ir, ³²P, ⁹⁰Sr, ²²⁶Ra and a combination thereof.
13. A method of treating the sequelae caused by exposure to a dose of ionizing radiation comprising the step of supplementing the mucosal immune system in a subject by orally administering an effective amount of a lactoferrin composition.
14. A method of enhancing a mucosal immune response in the gastrointestinal tract in a subject that received an absorbed dose of ionizing radiation comprising the step of orally administering an effective amount of a lactoferrin composition.
15. The method of sentence 14, wherein the lactoferrin composition stimulates the production of a cytokine or a chemokine.
16. The method of sentence 14, wherein the lactoferrin composition results in an inhibition of a cytokine or a chemokine.
17. The method of sentence 15, wherein the cytokine is selected from the group consisting of interleukin-18 (IL-18), interleukin-12 (IL-12), granulocyte/macrophage colony-stimulating factor (GM-CSF), and gamma interferon (IFN-γ).
18. The method of sentence 15, wherein the chemokine is macrophage inflammatory protein 3 alpha (MIP-3α), macrophage inflammatory protein 1 alpha (MIP-1α), macrophage inflammatory protein 1 beta (MIP-1β).
19. The method of sentence 16, wherein the cytokine is selected from the group consisting of interleukin-2 (IL-2), interleukin-4 (IL-4), interleukin-5 (IL-5), interleukin-10 (IL-10), and tumor necrosis factor alpha (TNF-α).
20. The method of sentence 33, wherein the lactoferrin composition inhibits the production of matrix metalloproteinases (MMPs).
21. The method of sentence 17, wherein interleukin-18 or granulocyte/macrophage colony-stimulating factor stimulates the production or activity of immune cells.
22. The method of sentence 21, wherein the immune cells are selected from the group consisting of T lymphocytes, natural killer cells, macrophages, dendritic cells, and polymorphonuclear cells.
23. The method of sentence 22, wherein the polymorphonuclear cells are neutrophils.
24. The method of sentence 22, wherein the T lymphocytes are selected from the group consisting of CD4+, CD8+ and CD3+ T cells.
25. A method of decreasing mortality of a subject that received an absorbed dose of ionizing radiation comprising the step of orally administering to said subject an effective amount of a lactoferrin composition to attenuate the effect of said absorbed dose.
26. A method of attenuating the damaging effects of an absorbed dose of irradiation in a subject comprising the step of orally administering to said subject an effective amount of a lactoferrin composition to attenuate the damaging effect of said absorbed dose.
27. The method of sentence 26, wherein attenuating the damage results in a decrease in morbidity of said subjects.
28. The method of sentence 26, wherein attenuating the damage results in a decrease in gut-associated systemic bacterial, viral or fungal infections.
29. The method of sentence 26, wherein attenuating the damage results in a decrease in mortality of said subjects.
30. A method of attenuating the damaging effects of an absorbed dose of irradiation in a subject comprising the step of orally administering to said subject an effective amount of a lactoferrin composition in combination with a radioprotective agent to attenuate the damaging effect of said absorbed dose.
31. The method of sentence 30, wherein the radioprotective agent is granulocyte-stimulating factor (G-CSF) (Filgrastim/(Neupogen)) or Amifostine.
32. A method of treating the sequelae caused by exposure to a dose of ionizing radiation comprising the step of supplementing the mucosal immune system in a subject by topically administering an effective amount of a lactoferrin composition.
33. A method of enhancing an immune response in the dermal tissues in a subject that received an absorbed dose of ionizing radiation resulting in radiation dermatitis comprising the step of topically administering an effective amount of a lactoferrin composition.
34. The method of sentence 33, wherein the lactoferrin composition stimulates the production of a cytokine or a chemokine.
35. The method of sentence 33, wherein the lactoferrin composition results in an inhibition of a cytokine or a chemokine.
36. The method of sentence 35, wherein the cytokine is selected from the group consisting of interleukin-18 (IL-18), interleukin-12 (IL-12), granulocyte/macrophage colony-stimulating factor (GM-CSF), and gamma interferon (IFN-γ).
37. The method of sentence 35, wherein the chemokine is macrophage inflammatory protein 3 alpha (MIP-3α), macrophage inflammatory protein 1 alpha (MIP-1α), macrophage inflammatory protein 1 beta (MIP-1β).
38. The method of sentence 35, wherein the cytokine is selected from the group consisting of interleukin-2 (IL-2), interleukin-4 (IL-4), interleukin-5 (IL-5), interleukin-10 (IL-10), and tumor necrosis factor alpha (TNF-α).
39. The method of sentence 33, wherein the lactoferrin composition inhibits the production of matrix metalloproteinases (MMPs).
40. The method of sentence 36, wherein interleukin-18 or granulocyte/macrophage colony-stimulating factor stimulates the production or activity of immune cells.
41. The method of sentence 40, wherein the immune cells are selected from the group consisting of T lymphocytes, natural killer cells, macrophages, dendritic cells, and polymorphonuclear cells.
42. The method of sentence 41, wherein the polymorphonuclear cells are neutrophils.
43. The method of sentence 41, wherein the T lymphocytes are selected from the group consisting of CD4+, CD8+ and CD3+ T cells.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawing, in which:

FIG. 1: shows the survival rates for mice exposed to a whole-body lethal dose of ionizing radiation of about 10 Gy. The dashed line indicates Talactoferrin treated mice and the solid line represents the placebo control mice.

FIG. 2: Treatment with talactoferrin accelerates recovery of lymphocytes in circulation depleted by irradiation. The chart shows FACS for the total number of white blood cells before irradiation and at various time points after whole-body non-lethal irradiation of mice with about 5 Gy. * Indicates an unpaired, two-tailed p value of 0.0359.

FIG. 3: shows mouse health status scores following 6 Gy irradiation. N=20 for this data set and the Placebo and Talactoferrin cohorts have significantly different end point values (p=0.0259).

### DETAILED DESCRIPTION OF THE INVENTION

It is readily apparent to one skilled in the art that various embodiments and modifications can be made to the invention disclosed in this Application without departing from the scope and spirit of the invention.

### I. DEFINITIONS

As used herein, the use of the word “a” or “an” when used in conjunction with the term “comprising” in the claims and/or the specification may mean “one,” but it is also consistent with the meaning of “one or more,” “at least one,” and “one or more than one.” Still further, the terms “having”, “including”, “containing” and “comprising” are interchangeable and one of skill in the art is cognizant that these terms are open ended terms.

The term “lactoferrin composition” as used herein refers to a composition having lactoferrin, a portion or part of lactoferrin, an N-terminal lactoferrin variant, or a combination thereof.

The term “lactoferrin” or “LF” as used herein refers to native or recombinant lactoferrin. Native lactoferrin can be obtained by purification from mammalian milk or colostrum or from other natural sources. Recombinant lactoferrin (rLF) can be made by recombinant expression or direct production in genetically altered animals, plants, fungi, bacteria, or other prokaryotic or eukaryotic species, or through chemical synthesis.

The term “human lactoferrin” or “hLF” as used herein refers to native or recombinant human lactoferrin. Native human lactoferrin can be obtained by purification from human milk or colostrum or from other natural sources. Recombinant human lactoferrin (rhLF) can be made by recombinant expression or direct production in genetically altered animals, plants, fungi, bacteria, or other prokaryotic or eukaryotic species, or through chemical synthesis.

The term “bovine lactoferrin” or “bLF” as used herein refers to native or recombinant bovine lactoferrin. Native bovine lactoferrin can be obtained by purification from bovine milk. Recombinant bovine lactoferrin (rbLF) can be made by recombinant expression or direct production in genetically altered animals, plants, fungi, bacteria, or other prokaryotic or eukaryotic species, or through chemical synthesis.

The term “N-terminal lactoferrin variant” as used herein refers to lactoferrin wherein at least the N-terminal glycine has been truncated and/or substituted. N-terminal lactoferrin variants also include, but are not limited to deletion and/or substitution of one or more N-terminal amino acid residues, for example 1, 2, 3, 4, 5, 6, 7, 8, 9, 10, 11, 12, 13, 14, 15, or 16 N-terminal amino acid residues, etc. Thus, N-terminal lactoferrin variants comprise at least deletions or truncations and/or substitutions of 1 to 16 N-terminal amino acid residues. The deletion and/or substitution of at least the N-terminal glycine of lactoferrin mediates the same biological effects as full-length lactoferrin and/or may enhance lactoferrin's biological activity, for example by stimulating the production of various cytokines (e.g., IL-18, MIP-3α, GM-CSF or IFN-γ) by inhibiting various cytokines, (e.g., IL-2, IL-4, IL-5, IL-10, or TNF-α, and by improving other parameters which promotes or enhances the well-being of the subject.

The term “oral administration” as used herein includes, but is not limited to oral, buccal, enteral or intragastric administration.

The term “immunocompromised” as used herein is defined as the status of a subject who is, at the time of exposure to potential pathogens unable completely and competently to respond to the pathogens due to the subject's reduced one or more mechanisms for normal defense against infection, the thus status being brought about by an exposure of the said subject to a damaging type and dose of ionizing radiation. More than one defect in the body's mechanism may be affected (e.g., bone marrow damage, depletion of blood lymphocytes, dendritic cells and other cells of the immune system, damage and consequent increase in permeability and hence a decrease in the protective function of the epithelium (e.g., of the gut, the skin, the lungs), etc.

The said “immunocompromised status” as used herein is the consequence of exposure to, and dose absorption by the body of damaging ionizing radiation of various types and strength. Ionizing radiation is a type of particle radiation in which an individual particle (for example, a photon, electron, or helium nucleus) carries enough energy to ionize an atom or molecule (that is, to completely remove an electron from its orbit). These ionizations, if enough occur, can be very destructive to living tissue. The composition of ionizing radiation can vary. Electromagnetic radiation can cause ionization if the energy per photon is high enough (that is, the wavelength is short enough). Far ultraviolet, X-rays, and gamma rays are all ionizing radiation. Ionizing radiation may also consist of fast-moving particles such as electrons, positrons, or small atomic nuclei.

The term “parenteral administration” as used herein includes any form of administration in which the compound is absorbed into the subject without involving absorption via the intestines. Exemplary parenteral administrations that are used in the present invention include, but are not limited to intramuscular, intravenous, intraperitoneal, intraocular, or intraarticular administration.

The term “pharmaceutically acceptable carrier” as used herein includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the vectors or cells of the present invention, its use in therapeutic compositions is contemplated. Supplementary active ingredients also can be incorporated into the compositions.

The term “pharmaceutical composition” as used herein refers to a lactoferrin composition that this dispersed in a pharmaceutically acceptable carrier. The lactoferrin composition can comprise lactoferrin or an N-terminal lactoferrin variant in which at least the N-terminal glycine amino acid residue is truncated or substituted.

The term “subject” as used herein, is taken to mean any mammalian subject to which a human lactoferrin composition is orally administered according to the methods described herein. In a specific embodiment, the methods of the present invention are employed to treat a human subject.

The term “therapeutically effective amount” as used herein refers to an amount that results in an improvement or remediation of the symptoms of the disease or condition.

The term “topical administration” as used herein includes, but is not limited to topical, dermal (e.g., trans-dermal or intra-dermal), epidermal, or subcutaneous.

The term “treating” and “treatment” as used herein refers to administering to a subject a therapeutically effective amount of a recombinant human lactoferrin composition so that the subject has an improvement in the disease. The improvement is any improvement or remediation of the symptoms. The improvement is an observable or measurable improvement. Thus, one of skill in the art realizes that a treatment may improve the disease condition, but may not be a complete cure for the disease.

### II. LACTOFERRIN

The lactoferrin used according to the present invention can be obtained through isolation and purification from natural sources, for example, but not limited to mammalian milk. The lactoferrin is preferably mammalian lactoferrin, such as bovine or human lactoferrin. In preferred embodiments, the lactoferrin is produced recombinantly using genetic engineering techniques well known and used in the art, such as recombinant expression or direct production in genetically altered animals, plants or eukaryotes, or chemical synthesis. See, i.e., U.S. Pat. Nos. 5,571,896; 5,571,697 and 5,571,691, which are herein incorporated by reference.

In certain aspects, the present invention provides lactoferrin variants having enhanced biological activities of natural LF and or rLF, e.g., the ability to stimulate and/or inhibit cytokines or chemokines. In particular, the invention provides variants of lactoferrin from which at least the N-terminal glycine residue has been substituted and/or truncated. The N-terminal lactoferrin variants may occur naturally or may be modified by the substitution or deletion of one or more amino acids.

The deletional variants can be produced by proteolysis of lactoferrin and/or expression of a polynucleotide encoding a truncated lactoferrin as described in U.S. Pat. No. 6,333,311, which is incorporated herein by reference.

Substitutional variants or replacement variants typically contain the exchange of one amino acid for another at one or more sites within the protein. Substitutions can be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

Each amino acid has been assigned a hydropathic index on the basis of their hydrophobicity and charge characteristics (Kyte and Doolittle, 1982), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those that are within ±1 are particularly preferred, and those within ±0.5 are even more particularly preferred.

It is also understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine −0.5); cysteine (−1.0); methionine (−1.3); valine (−1.5); leucine (−1.8); isoleucine (−1.8); tyrosine (−2.3); phenylalanine (−2.5); tryptophan (−3.4).

Still further, it is understood that an amino acid can be substituted for another having a similar hydrophilicity value and still obtains a biologically equivalent and immunologically equivalent protein. In such changes, the substitution of amino acids whose hydrophilicity values are within ±2 is preferred, those that are within ±1 are particularly preferred, and those within ±0.5 are even more particularly preferred.

Thus, in the present invention, substitutional variants or replacement can be produced using standard mutagenesis techniques, for example, site-directed mutagenesis as disclosed in U.S. Pat. Nos. 5,220,007; 5,284,760; 5,354,670; 5,366,878; 5,389,514; 5,635,377; 5,789,166, and 6,333,311, which are incorporated herein by reference. It is envisioned that at least the N-terminal glycine amino acid residue can be replaced or substituted with any of the twenty natural occurring amino acids, for example a positively charged amino acid (arginine, lysine, or histidine), a neutral amino acid (alanine, asparagine, cysteine, glutamine, glycine, isoleucine, leucine, methionine, phenylaline, proline, serine, threonine, tryptophan, tyrosine, valine) and/or a negatively charged amino acid (aspartic acid or glutamic acid). Still further, it is contemplated that any amino acid residue within the range of N1 to N16 can be replaced or substituted. It is envisioned that at least up to 16 of the N-terminal amino acids residues can be replaced or substituted as long as the protein retains it biological and/or functional activity, which is stimulating the production of various cytokines, (e.g., IL-18, MIP-3α, GM-CSF or IFN-γ) by inhibiting various cytokines, (e.g., IL-2, IL-4, IL-5, IL-10, and TNF-α) and/or by improving the parameters related to which promotes or enhances the well-being of the subject with respect to its medical conditions. Thus, the N-terminal lactoferrin variants of the present invention are considered functional equivalents of lactoferrin.

In terms of functional equivalents, it is well understood by the skilled artisan that, inherent in the definition of a “biologically functional equivalent” protein is the concept that there is a limit to the number of changes that may be made within a defined portion of the molecule while retaining a molecule with an acceptable level of equivalent biological activity and/or enhancing the biological activity of the lactoferrin molecule. Biologically functional equivalents are thus defined herein as those proteins in which selected amino acids (or codons) may be substituted. Functional activity is defined as the ability of lactoferrin to stimulate or inhibit various cytokines or chemokines and/or by improving the parameters which promote or enhance the well-being of the subject with respect to its medical conditions. For example, extension of the subject's life by any period of time; attenuation of damage due to radiation; accelerated normalization of the subject's compromised immune system; a decrease in pain to the subject that can be attributed to the subject's condition.

Still further, the N-terminal amino acid residues can be substituted with a modified and/or unusual amino acids. A table of exemplary, but not limiting, modified and/or unusual amino acids is provided herein below.

TABLE 5
Modified and/or Unusual Amino Acids

| Abbr. | Amino Acid | Abbr. | Amino Acid |
|---|---|---|---|
| Aad | 2-Aminoadipic acid | EtAsn | N-Ethylasparagine |
| BAad | 3-Aminoadipic acid | Hyl | Hydroxylysine |
| BAla | beta-alanine, beta-Amino- | AHyl | allo-Hydroxylysine |
| | propionic acid | | |
| Abu | 2-Aminobutyric acid | 3Hyp | 3-Hydroxyproline |
| 4Abu | 4-Aminobutyric acid, | 4Hyp | 4-Hydroxyproline |
| | piperidinic acid | | |
| Acp | 6-Aminocaproic acid | Ide | Isodesmosine |
| Ahe | 2-Aminoheptanoic acid | Aile | allo-Isoleucine |
| Aib | 2-Aminoisobutyric acid | MeGly | N-Methylglycine, sarcosine |
| BAib | 3-Aminoisobutyric acid | MeIle | N-Methylisoleucine |
| Apm | 2-Aminopimelic acid | MeLys | 6-N-Methyllysine |
| Dbu | 2,4-Diaminobutyric acid | MeVal | N-Methylvaline |
| Des | Desmosine | Nva | Norvaline |
| Dpm | 2,2′-Diaminopimelic acid | Nle | Norleucine |
| Dpr | 2,3-Diaminopropionic acid | Orn | Ornithine |
| EtGly | N-Ethylglycine | | |

The presence and the relative proportion of an N-terminal lactoferrin variants (deletions and/or substitutions) in a preparation of lactoferrin (lactoferrin composition) may be done by determination of the N-terminal amino acid sequence by the process of Edman degradation using standard methods. A relative proportion of N-terminal lactoferrin variant comprises at least 1% of the lactoferrin composition, at least 5% of the lactoferrin composition, at least 10% of the lactoferrin composition, at least 25% of the lactoferrin composition, at least 50% of the lactoferrin composition or any range in between.

In this method, the protein is reacted with phenylisothiocyanate (PITC), which reacts with the amino acid residue at the amino terminus under basic conditions to form a phenylthiocarbamyl derivative (PTC-protein). Trifluoroacetic acid then cleaves off the first amino acid as its anilinothialinone derivative (ATZ-amino acid) and leaves the new amino terminus for the next degradation cycle.

The percentage of N-terminal lactoferrin variant may also be done more precisely by using a Dansylation reaction. Briefly, protein is dansylated using Dansyl chloride reacted with the protein in alkaline conditions (pH 10). Following the Dansylation, the reaction mixtures are dried to pellets, then completely hydrolyzed in 6N HCl. The proportion of N-terminal amino acids are identified by RP HPLC using an in-line fluorometer in comparison with standards made up of known dansylated amino acids.

### III. PHARMACEUTICAL COMPOSITIONS

The present invention is drawn to a composition comprising lactoferrin that is dispersed in a pharmaceutical carrier. The lactoferrin that is contained in the composition of the present invention comprises lactoferrin or an N-terminal lactoferrin variant in which at least the N−1 terminal glycine residue is truncated or substituted. N-terminal lactoferrin variants include variants that at least lack the N-terminal glycine residue or contain a substitution at the N-terminal glycine residue. The substitution can comprise substituting a natural or artificial amino acid residue for the N-terminal glycine residue. For example, the substitution can comprise substituting a positive amino acid residue or a negative amino acid residue for the N-terminal glycine residue or substituting a neutral amino acid residue other than glycine for the N-terminal glycine residue. Other N-terminal lactoferrin variants include lactoferrin lacking one or more N-terminal residues or having one or more substitutions in the N-terminal. The N-terminal lactoferrin variant comprises at least 1% of the composition, at least 5% of the composition, at least 10% of the composition, at least 25% of the composition, at least 50% of the composition or any range in between.

Further in accordance with the present invention, the composition of the present invention suitable for administration is provided in a pharmaceutically acceptable carrier with or without an inert diluent. The carrier should be assimilable and includes liquid, semi-solid, e.g., pastes, or solid carriers. Except insofar as any conventional media, agent, diluent or carrier is detrimental to the recipient or to the therapeutic effectiveness of the composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof.

In accordance with the present invention, the composition is combined with the carrier in any convenient and practical manner, e.g., by solution, suspension, emulsification, admixture, encapsulation, absorption and the like. Such procedures are routine for those skilled in the art.

In a specific embodiment of the present invention, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, e.g., denaturation in the stomach. Examples of stabilizers for use in the composition include buffers, amino acids such as glycine and lysine, carbohydrates such as dextrose, mannose, galactose, fructose, lactose, sucrose, maltose, sorbitol, mannitol, etc., proteolytic enzyme inhibitors, and the like. The composition for oral administration which is combined with a semi-solid or solid carrier can be further formulated into hard or soft shell gelatin capsules, tablets, or pills. More preferably, gelatin capsules, tablets, or pills are enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the lactoferrin composition to be released and absorbed by specialized cells, e.g., epithelial enterocytes and Peyer's patch M cells.

In another embodiment, a powdered composition is combined with a liquid carrier such as, e.g., water or a saline solution, with or without a stabilizing agent. The topical embodiment may include formulating excipients such as Carbopol, poly(ethylene glycol), preservatives, etc.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Administration of the lactoferrin compositions according to the present invention will be via any common route, orally, parenterally, or topically. Exemplary routes include, but are not limited to oral, nasal, buccal, rectal, vaginal, parenteral, intramuscular, intraperitoneal, intravenous, intraarterial, intratumoral, or dermal. Such compositions would normally be administered as pharmaceutically acceptable compositions as described herein.

The amount of administered lactoferrin in the present invention may vary from about 0.01 to 2.0 g/kg, preferably from 0.01 to 0.5 g/kg, as a single or a divided dose. In preferred embodiments, the composition of the present invention comprises a lactoferrin concentration of about 0.1% to about 100%, in a solid, semi-solid (gel) or liquid formulation. The lactoferrin composition may comprise lactoferrin or an N-terminal lactoferrin variant in which at least the N−1 terminal glycine residue is truncated and/or substituted.

Upon formulation, solutions are administered in a manner compatible with the dosage formulation and in such amount as is therapeutically effective to result in an improvement or remediation of the symptoms. The formulations are easily administered in a variety of dosage forms such as ingestible solutions, drug release capsules, dermal ointments and the like. Some variation in dosage can occur depending on the condition of the subject being treated. The person responsible for administration can, in any event, determine the appropriate dose for the individual subject. Moreover, for human administration, preparations meet sterility, general safety and purity standards as required by FDA Office of Biologics standards.

### IV. TREATMENT

In accordance with the present invention, a lactoferrin composition provided in any of the above-described pharmaceutical carriers is orally or topically administered to a subject suspected of or having been exposed to irradiation or administered to a subject prior to exposure to irradiation. One of skill in the art can determine the therapeutically effective amount of lactoferrin to be administered to a subject based upon several considerations, such as absorption, metabolism, method of delivery, age, weight, severity of ionizing damage and response to the therapy. Oral administration of the lactoferrin composition includes oral, buccal, enteral or intragastric administration. It is also envisioned that the composition may be used as a food additive. For example, the composition is sprinkled on food or added to a liquid prior to ingestion. Topical administration of the lactoferrin composition includes topical, dermal, epidermal, or subcutaneous administration.

Treatment regimens may vary as well, and often depend on the type of ionizing damage or exposure, location of damage or exposure, disease progression that resulted from damage or exposure, and health and age of the patient. Obviously, certain types of conditions will require more aggressive treatment, while at the same time, certain patients cannot tolerate more taxing protocols. The clinician will be best suited to make such decisions based on the known efficacy and toxicity (if any) of the therapeutic formulations.

The lactoferrin composition can be administered orally as a solution in a suitable buffer, or as a solid oral dosage in the form of a capsule, tablet or similar suitable format, or as a topical formulation. The amount of lactoferrin that is administered is from 0.01 to 2.0 g/kg, preferably from 0.01 to 1.0 g/kg, as a single or a divided dose. The treatment is envisaged to continue until the damage has been normalized, preferably for 30 days of continuous treatment. The effect of treatment can be monitored by determining peripheral blood cell composition, in particular the content of white blood cells in circulation, and more generally by the overall physical status of the subjects.

The treatments may include various “unit doses.” Unit dose is defined as containing a predetermined quantity of the therapeutic composition (lactoferrin composition) calculated to produce the desired responses in association with its administration, i.e., the appropriate route and treatment regimen. The quantity to be administered, and the particular route and formulation, are within the skill of those in the clinical arts. Also of import is the subject to be treated, in particular, the state of the subject and the protection desired. A unit dose administered i.v. or s.c. need not be administered as a single injection but may comprise continuous infusion over a set period of time.

In specific embodiments, lactoferrin composition is given in a single dose or multiple doses. The single dose may be administered daily, or multiple times a day, or multiple times a week. In a further embodiment, the lactoferrin composition is given in a series of doses. The series of doses may be administered daily, or multiple times a day, weekly, or multiple times a week.

In a preferred embodiment of the present invention, lactoferrin composition is administered in an effective amount to prevent, reduce, decrease, or inhibit the damage caused by irradiation of the body by damaging ionizing radiation and improve patient survival. The amount of lactoferrin that is administered is from 0.01 to 2.0 g/kg, preferably from 0.01 to 0.5 g/kg, as a single or a divided dose. The treatment is envisaged to continue until the damage has been normalized, preferably for 30 days of continuous treatment.

The improvement is any observable or measurable change for the better. The composition and the method of treatment of this invention may decrease the mortality of subjects exposed to damaging irradiation. In other aspect, the composition of this invention is administered in an effective amount to decrease, reduce, inhibit, prevent or eliminate damage to, and the loss of function of the cells of the immune system, and the loss of function of the primary physical means of body defense, for example the GI epithelial barrier. Repeated administration of lactoferrin composition can result in the attenuation of the consequences of absorption by the body of a damaging dose of radiation.

In certain embodiments, it is envisioned that the immune system, whether local, systemic or mucosal, is enhanced by the lactoferrin composition stimulating cytokines and/or chemokines. Exemplary cytokines include interleukin-18 and GM-CSF in the gastrointestinal tract, which are known to enhance immune cells or stimulate production of immune cells. For example, interleukin-18 enhances natural killer cells or T lymphocytes, which can kill bacteria infecting a wound. In specific embodiments, interleukin-18 (IL-18) enhances CD4+, CD8+ and CD3+ cells. It is known by those of skill in the art that IL-18 is a Th1 cytokine that acts in synergy with interleukin-12 and interleukin-2 in the stimulation of lymphocyte IFN-gamma production. Other cytokines or chemokines may also be enhanced for example, but not limited to IL-12, IL-1b, MIP-3α, MIP-1α or IFN-gamma. Other cytokines or enzymes may be inhibited for example, but not limited to IL-2, IL-4, IL-5, IL-10, TNF-α, or matrix metalloproteinases.

Damage to the immune and hematopoietic system following an absorbed dose of damaging irradiation makes subjects susceptible to opportunistic infections and disease. Total leukocyte count is traditionally as an indicator of immune system damage. While all PBMCs (Peripheral Blood Mononuclear Cells) decline in absolute numbers after radiation exposure, some change faster than others, leading to alterations in the proportions of various blood cell populations relative to their original proportions. Thus, it is envisioned that the lactoferrin composition of the present invention can enhance or increase the PBMCs or reduce the attenuation of PBMCs. More specifically it is known that the damage results in changes in the relative composition of immune cells in circulation such as an increase in CD4+ T lymphocytes, decrease in B lymphocytes and a dramatic increase in natural killer cells. Such changes result in immune dysregulation and depressed immune responsiveness to antigenic challenge. Thus, the lactoferrin composition of the present invention can correct or positively alter the immune dysregulation that occurs in response to irradiation damage.

In further embodiments, cytokines, for example, interleukin-18 or granulocyte/macrophage colony-stimulating factor, can stimulate the production or activity of immune cells. The immune cells include, but are not limited to T lymphocytes, natural killer cells, macrophages, dendritic cells, and polymorphonuclear cells. More specifically, the polymorphonuclear cells are neutrophils and the T lymphocytes are selected from the group consisting of CD4+, CD8+ and CD3+ T cells.

Still further, it is envisioned that lactoferrin composition stimulates production of MIP-3alpha from hepatocytes. Lactoferrin is known to contribute to the defense systems of the body through its anti-microbial properties. In addition, evidence suggests that recombinant human lactoferrin (rhLF) elicits a more general innate-like immune response when administered orally. The innate immune system is the ‘first line of defense’ of the body against hostile environments and comprise of a variety of effector and cellular mechanisms. This innate immune response is initially likely mediated by the ‘detection system’ of receptors known to be present on the surface of the gut epithelial cells, such as pattern recognition receptors, IL-1 receptor and general ‘scavenger’ receptors. These receptors recognize and respond to specific structural features of the presented molecules. As a result, various intracellular signaling pathways may be initiated (e.g., NFκB, Wnt, etc.) that result in the overall orchestration of the cellular response of the body to the prevailing biological situation (e.g., infection). RhLF and compositions derived from rhLF elicit a similar response of human hepatocytes in vitro in terms of producing an important chemokine—namely MIP-3-alpha.

Further, when applied orally, the effect of lactoferrin on maintaining the integrity of the GI barrier is also very relevant as this leads to attenuating the process of translocation of bacteria and endotoxin across the GI epithelium. Thus, lactoferrin reduces the likelihood of development of serious systemic infections following irradiation. Additionally, lactoferrin may also to reduce the overall microbial burden of the gut and to reduce the amount of free endotoxin (LF binds endotoxin) and reduces the extent of translocation of these ‘undesirables.’

### V. COMBINATION TREATMENT

In order to increase the effectiveness of the lactoferrin composition of the present invention, it may be desirable to combine the composition of the present invention with other agents effective in providing protection or treating ionizing radiation. These other radioprotective compositions would be provided in a combined amount effective to promote therapeutic benefit. This process may involve administering the lactoferrin composition of the present invention and the agent(s) or multiple factor(s) at the same time. This may be achieved by administering a single composition or pharmacological formulation that includes both agents, or by administering two distinct compositions or formulations, at the same time, or at times close enough so as to result in an overlap of this effect, wherein one composition includes the lactoferrin composition and the other includes the second agent(s).

Alternatively, the lactoferrin composition of the present invention may precede or follow the other radioprotective agent and/or treatment by intervals ranging from minutes to weeks. In embodiments where the radioprotective agent and lactoferrin composition are administered or applied separately, one would generally ensure that a significant period of time did not expire between the time of each delivery, such that the agent and lactoferrin composition would still be able to exert an advantageously combined effect. In such instances, it is contemplated that one may contact the area and/or administer to the subject to be treated both modalities within about 1-14 days of each other and, more preferably, within about 12-24 hours of each other. In some situations, it may be desirable to extend the time period for treatment significantly, however, where several days (2, 3, 4, 5, 6 or 7) to several weeks (2, 3, 4, 5, 6, 7 or 8) lapse between the respective administrations.

In specific embodiment, treatment with lactoferrin can be combined with other treatments aiming to lessen the effects of damaging radiation, for example with granulocyte-stimulating factor (G-CSF) (Filgrastim/(Neupogen)) or with Amifostine, or with other agents intended to treat the consequences of radiation damage.

A. Thiol Containing Compounds

Examples of thiols that can be used as radioprotective agents include, but are not limited to cysteine, cysteamine, cystamine, AET and 2-mercaptoethylguanidine (MEG). The sulfhydrylamines are also potent agents which reduce temperatures and physiological pH. The dose reduction factor (DRF) of various compounds ranges from 1.4 to 2.0. This class of compounds is characterized by the sulfhydryl compounds (SH) and amine (NH₂) separated by 2 carbon atoms.

Other —SH radicals that can be used as radioprotective agents include, but are not limited to thiourea, thiouracil, dithiocarbamate, dithioxamides, thiazolines, sulfoxides and sulfones.

B. Pharmacological Agents

Pharmacological agents that can be used as radioprotective agents can include anesthetic drugs and alchohol, analgesics (e.g., morphine, heroin, sodium salicylate) tranquilizers, cholinergic drugs (e.g., acetylcholine, metacholilne), epinephrine and norepinephrine, dopamine, histamine, serotonin, glutathione, vitamin C, vitamin E, and hormones (e.g., estrogen).

C. Other Agents

Other radioprotective agents can include, but are not limited to cyanide, derivatives of nucleic acids (e.g., ATP), sodium fluoracetate, para-aminopropiophenone (PAPP), mellitin, endotoxins, imidazole, adenosine 3′,5′-cyclic monophosphate (cAMP), antibiotics, lipids (e.g. olive oil), erythropoietin, carbon monoxide (competes with hemoglobulin), hydrochloric mercaptoethylamine (MEA), sodium hydrogen S-(2-aminoethyl) phosphorothioic acid (WR-638), S-2-(3-aminopropylamino)ethyl phosphorothioic acid (WR-2721) S-2-(3-aminopropylamino) propylphosphorothioic acid (WR-44923), natural polyamines putrescine (1,4-Diaminobutane), spermidine and spermine.

Other radioprotectors can include, but are not limited to nitroxide Tempol (4-hydroxy-2,2,6,6,-tetramethylpiperidine-1-oxyl), calcium antagonists (diltiazem, nifedipine and nimodipine), stobadine and bacterial endotoxins.

D. Immunomodulators

Immunomodulators are another class of radioprotectors that can enhance survival in irradiated animals. The most extensively studied cytokines regarding their radioprotective action are: interleukin-1 (IL-1), tumor necrosis factor alpha (TNF-α), granulocyte colony-stimulating factor (G-CSF) and granulocyte-macrophage CSF (GM-CSF).

Another immunomodulator that is a radioprotective agent is AS101 (ammonium trichloro(dioxyethylene-O—O′) Tellurate) which stimulates the production of a variety of cytokines and presents radioprotective activity in mice.

### VI. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Effect of Orally Administered Lactoferrin on Radiation-Induced Mortality in Mice

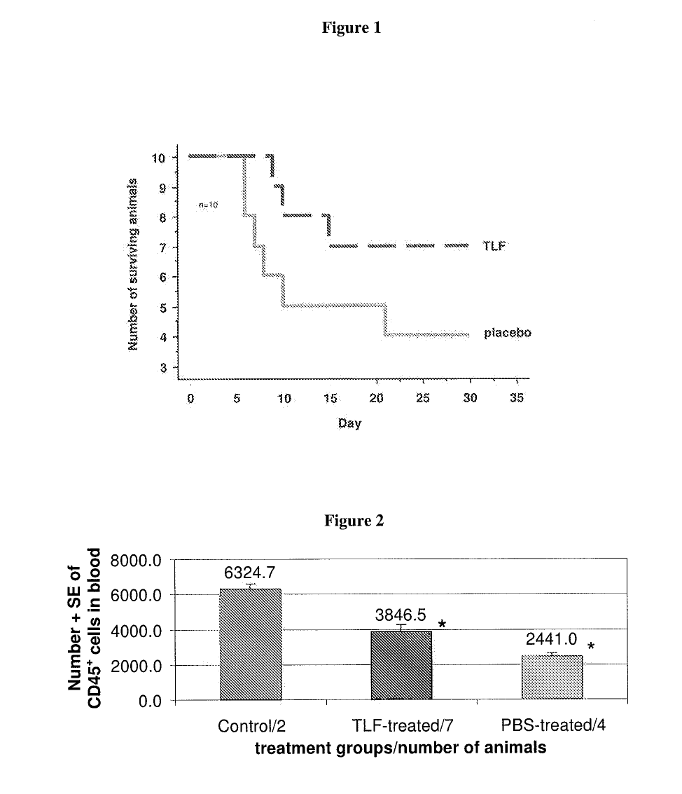

Mice (n=10) were exposed to a whole-body lethal dose of ionizing radiation of about 10 Gy. Immediately after irradiation, mice were treated by oral gavage with lactoferrin at a dose of 2.9 g/m² or with vehicle placebo. This dose was administered to each mouse once a day for 30 days after exposure. At day 30, there were 4 surviving mice in the placebo group and 7 surviving mice in the TLF-treated group, i.e., 75 relative % increased survival due to TLF treatment (FIG. 1). In addition, TLF-treated mice showed better clinical signs throughout the study.

### Example 2

### Effect of Orally Administered Lactoferrin on the Recovery of White Blood Cells in Circulation Following Exposure to Damaging Irradiation in Mice

Mice (n=10) were exposed to a whole-body non-lethal dose of ionizing radiation of about 5 Gy. Immediately after irradiation, mice were treated by oral gavage with lactoferrin at a dose of 2.9 g/m² or with vehicle placebo. This dose was administered to each mouse once a day for 34 days after exposure. Non-irradiated, untreated mice were used as control. Samples of blood from mice were analyzed by FACS before irradiation and at various time points after irradiation for the total number of white blood cells. It was found that treatment with TLF, as compared to treatment with a vehicle, in addition to improving mice survival, accelerated the rate of recovery of the number of white blood cells in circulation (see FIG. 2). Such improved recovery is likely to result in a higher resistance of mice to secondary infections.

### Example 3

### Effect of Orally Administered Lactoferrin on Radiation-Induced Damage in Mice

Mice (n=12) were exposed to a whole-body non-lethal dose of ionizing radiation of about 5 Gy. Immediately after irradiation, mice (n=6) were treated by oral gavage with lactoferrin at a dose of 2.9 mg/m² or with vehicle placebo (n=6). This dose was administered to each mouse once a day for 30 days after exposure. The blood was collected from mice at various time intervals and the cellular composition of mice blood is analyzed. The number of cells of the immune system normalized faster in the lactoferrin-treated mice compared to placebo-treated mice (Table 1).

TABLE 1

| | |
|---|---|
| | Number of lymphocytes (as |
| Time after exposure | % of mononuclear cells) |

| [days] | Placebo-treated | LF-treated |
|---|---|---|
| 0 (before irradiation) | 60 | 60 |
| 1 | 15 | 15 |
| 7 | 22 | 30 |
| 14 | 30 | 50 |
| 28 | 40 | 63 |

### Example 4

### Effect of Orally Administered Lactoferrin on the Health Status and Mortality After Irradiation

Mice (n=20) were exposed to a whole-body 6 Gy dose of ionizing radiation. Immediately after irradiation, mice were treated by oral gavage with either talactoferrin (2.9 mg/kg) or with placebo. The dose was administered to each mouse once a day for 30 days following exposure. Talactoferrin increased the survival of irradiated mice by 50 relative % (i.e. twice as many (6) mice survived in the TLF-treated group as compared to the placebo group (3) at the end of the study). During the study, the health status of mice was evaluated daily, prior to dosing, using the approach of Morton (Morton 1999). A single numerical score of the health status was determined using the following parameters.

Activity 1—normal; 2—reduced; 3—low
Hunched posture 1—normal; 2—moderate, 3—extreme
Ruffled fur 1—normal; 2—slight; 3—moderate; 4—extreme
Breathing 1—normal; 2—laboured; 3—shallow; 4—rapid
Alertness 1—normal; 2—reduced; 3—low
Body weight 1—increased; 2—decreased
Dehydration 1—normal; 2—moderate, 3—extreme

### Diarrhea 1—no; 2—yes

Polyurea (wetness) 1—no; 2—yes

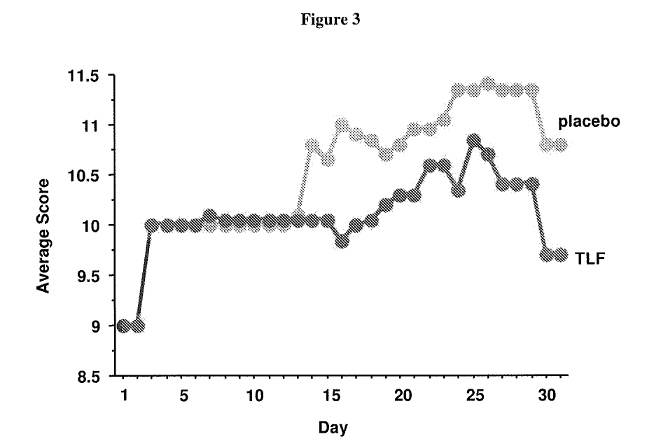

The above status score ranges from 9 to 26. Animals that died were given the final score of 30. The results are presented graphically in FIG. 3. Statistics were calculated using a repeated measure ANOVA. These results demonstrate a statistically significant improvement in the health scores of the talactoferrin group versus placebo (p=0.0259) following 6 Gy irradiation.

### Example 5

### Dose-Dependent Protection by Oral and Intravenous Lactoferrin Against Radiation-Induced Death in Mice

Mice (n=20/group) are exposed to a whole-body lethal dose of ionizing radiation of about 10 Gy. Immediately after irradiation, mice are treated by oral gavage with, or i.v. infusion of, lactoferrin at doses of 0 (vehicle), 0.19, 0.86, 2.0 and 2.9 mg/m². The mice are dosed once a day for 30 days after exposure. The effect of lactoferrin on the mortality of mice due to exposure to a lethal dose of ionizing radiation, and their overall health status are evaluated. Mortality and/or health status are improved in lactoferrin treated animals relative to control animals.

### Example 6

### Dose-Dependent Protection by Oral and Intravenous Lactoferrin Against Radiation-Induced Damage in Mice

Mice (n=6/group) are exposed to a whole-body non-lethal dose of ionizing radiation of about 5 Gy. Immediately after irradiation, mice are treated by oral gavage with, or i.v. infusion of, lactoferrin at doses of 0 (vehicle), 0.19, 0.86, 2.0, 2.9, and 5.8 mg/m². The doses are administered to each mouse once a day for 30 days after exposure. The blood is collected from mice at various time intervals and the cellular composition of mice blood is analyzed. The effect of lactoferrin on the mortality of mice due to exposure to ionizing radiation, on their blood composition, and their overall health status are evaluated. Mortality, blood cell recovery and/or health status are improved in lactoferrin treated animals relative to control animals.

### Example 7

### Efficacy of Oral and Intravenous Lactoferrin Administered by Different Regimens in Radiation-Induced Damage

Mice (n=6/group) are exposed to a whole-body non-lethal dose of ionizing radiation of about 5 Gy. Mice are treated with oral or i.v. lactoferrin 24 hours before irradiation, and then immediately after irradiation. In various groups of mice, the animals are treated a) twice a day with 1.45 mg/m² or 2.9 mg/m² doses, b) once a day with 2.9 mg/m² or 5.8 mg/m² c) every other day with 2.9 mg/m² or 5.8 mg/m² doses, or d) once a week with 2.9 mg/m² or 5.8 mg/m² doses of lactoferrin. The treatments are continued for 30 days after exposure. The blood is collected from mice at various time intervals and the cellular composition of mice blood is analyzed. The effect of lactoferrin on the mortality of mice due to exposure to ionizing radiation, on their blood composition, and their overall health status are evaluated. Mortality, blood cell recovery and/or health status are improved in lactoferrin treated animals relative to control animals.

### Example 8

### Efficacy of Oral and Intravenous Lactoferrin Administered by Different Regimens in Radiation-Induced Death

Mice (n=10/group) are exposed to a whole-body lethal dose of ionizing radiation of about 10 Gy. Mice are treated with oral or i.v. lactoferrin 24 hours before irradiation, and then immediately after irradiation. In various groups of mice, the animals are treated a) twice a day with 1.45 mg/m² or 2.9 mg/m² doses, b) once a day with 2.9 mg/m² or 5.8 mg/m², c) every other day with 2.9 mg/m² or 5.8 mg/m² dose, and d) once a week with a dose of 2.9 mg/m² or 5.8 mg/m² of lactoferrin. The treatments are continued for 30 days after exposure. The effect of different doses and dosage regimens of lactoferrin on the mortality of mice due to exposure to a lethal dose of ionizing radiation, and their overall health status are evaluated. Mortality and/or health status are improved in lactoferrin treated animals relative to control animals.

### Example 9

### Protective Effect of Oral and Intravenous Lactoferrin to Secondary Infection in Mice Following a Sub-Lethal Dose of Ionizing Irradiation

Mice (n=10/group) are exposed to a whole-body sub-lethal dose of ionizing radiation of about 5 Gy. Immediately after irradiation, mice are treated by oral gavage with, or i.v. infusion of, lactoferrin at a dose of 2.9 mg/m² or 5.8 mg/m² or with a placebo. Lactoferrin is administered to each mouse once a day for 30 days after exposure. Three (3) days after irradiation, mice are inoculated with a dose of ˜10¹² CFU/kg of enterotoxigenic E. coli by gastric gavage. The effect of different doses of lactoferrin on the mortality of mice exposed to ionizing radiation and an infectious organism, and the mouse overall health status are evaluated. Mortality and/or health status are improved in lactoferrin treated animals relative to control animals.

### Example 10

### Effect of Orally and Intravenously Administered Lactoferrin from Different Sources on Radiation-Induced Damage in Mice

Mice (n=24) are exposed to a whole-body non-lethal dose of ionizing radiation of about 5 Gy. Immediately after irradiation, mice (n=6 for each group of lactoferrin) are treated by oral gavage with, or i.v. infusion of, various lactoferrin compositions (different sources of human and bovine lactoferrin—Agennix, Ventria, Jarrow and Pharming) at a dose of 2.9 mg/m² or with vehicle placebo (n=6). This dose is administered to each group once a day for 30 days after exposure. The blood is collected from mice at various time intervals and the cellular composition of mice blood is analyzed. The effect of human and bovine lactoferrin from different sources on the mortality of mice due to exposure to a lethal dose of ionizing radiation, their blood composition and their overall health status are evaluated. Mortality, blood cell recovery and/or health status are improved in lactoferrin treated animals relative to control animals.

### Example 11

### Protection by Oral Lactoferrin Against Radiation-Induced Death in Beagle Dogs

Ten beagles of either sex, at a median age of 9 (range, 7 to 32) months are employed in this study. Five beagles receive TLF and no total-body irradiation (TBI). Groups of five beagles receive 400 cGy TBI and, within 2 hours, are given TLF at lactoferrin doses of 0.2, 0.4 or 0.8 g/m².

Dogs are quarantined on arrival, screened for evidence of disease, and observed for a minimum of 1 month before being released for use. They are de-wormed and vaccinated for rabies, distemper, leptospirosis, hepatitis, and parvovirus. Beagles are housed in an American Association for Accreditation of Laboratory Animal Care accredited facility in standard indoor runs, and provided commercial dog chow and chlorinated tap water ad libitum. Animal holding areas are maintained at 70±2° F. with 50%-10% relative humidity, using at least 15 air changes per hour of 100% conditioned fresh air. The dogs are on a 12-hour light/dark full-spectrum lighting cycle with no twilight. The protocol for this study is approved by the Institutional Animal Care and Use Committee.

All dogs receive 400 cGy TBI at 10 cGy/min from two opposing 60Co sources. The day of TBI is designated day 0. Hematocrit, reticulocyte, leukocyte, platelet, and differential counts are obtained before and daily after TBI. Necropsies with histologic examinations are performed routinely on all dogs that die.

Daily peripheral blood cell counts are plotted on a logarithmic scale versus time. For dogs that receive radiation, the means of the log blood counts for each day are calculated for the LF-treated and for control dogs. Graphically, these results are displayed with cubic spline curves connecting the daily means for each group to show mean log blood count as a function of time. Blood count profiles after TBI in the LF-treated and control groups are compared by modeling mean log blood counts as a 5th degree polynomial in time with a constant difference between group means, and tested whether this constant is significantly different from zero using the generalized estimating equation (GEE) technique with independent working covariance matrix. Blood count nadirs are calculated for dogs that survive at least 18 days. Testing for differences among the two groups in nadirs of platelet and neutrophil counts is performed using the Kruskal-Wallis test. Blood cell recovery is improved in lactoferrin treated animals relative to control animals.

### Example 12

### Protection by Oral Lactoferrin Against Radiation-Induced Death in Non-Human Primates

Male rhesus monkeys, Macaca mulatta, mean weight 4.35±0.32 kg, are housed in individual stainless steel cages in conventional holding rooms in animal facilities accredited by the American Association for Accreditation of Laboratory Animal Care. Monkeys are provided 10 air changes/hour of 100% fresh air, conditioned to 72°±2° F. with a relative humidity of 50%±20% and maintained on a 12-hour light/dark full spectrum light cycle, with no twilight. Monkeys are provided with commercial primate chow, supplemented with fresh fruit and tap water ad libitum. Experiment is conducted according to the principles enunciated in the Guide for the Care and Use of Laboratory Animals, prepared by the Institute of Laboratory Animal Resources, National Research Council.

Monkeys, following a pre-habituation period, are unilaterally irradiated in Lucite® restraining chairs with 250 kVp x-radiation at 13 cGy/minute in the posterior-anterior position, rotated 180° at the mid-dose (300 cGy) to the anterior-posterior position for completion of the total 600 cGy midline tissue exposure. Dosimetry is performed using paired 0.5 cm3 ionization chambers, with calibration factors traceable to the National Institute of Standards and Technology.

Using two experimental groups of 5 animals each, animals are irradiated at day 0 and randomly assigned to a treatment protocol: A) controls (n=5) receive orally vehicle (PBS) control and B) LF administered orally at 1.5 mg/m² once a day for 30 days. Complete blood counts are monitored for 40 days following irradiation and the durations of neutropenia (ANC<500/μl) and thrombocytopenia (PLT<20,000/μl) are assessed. Peripheral blood is obtained from the saphenous vein to assay complete blood (Sysmex K-4500; Long Grove, Ill.) and differential counts (Wright-Giemsa Stain, Ames Automated Slide Stainer; Elkhart, Ind.) for 40 days post-TBI.

All animals receive clinical support that consists of antibiotics and fluids as needed. Gentamicin (Elkin Sinn, an AH Robbins subsidiary; Chemy Hill, N.J.) (10 mg/day, i.m., qd) is administered during the first 7 days of treatment, and BaytrilR (Bayer Corporation; Shawnee Mission, Kans.; http://www.bayerus.com) (10 mg/day i.m., qd) is administered for the entire period of antimicrobial treatment. The administration of antibiotics continues until the animals maintain a WBC≧1,000/μl for 3 consecutive days and have attained an ANC≧500/μl. Blood cell recovery and/or health status are improved in lactoferrin treated animals relative to control animals.

### Example 13

### Protection by Oral Lactoferrin Against Radiation-Induced Death in Non-Human Primates

Non-human primates (n=10/group) are exposed to a whole-body lethal dose of ionizing radiation of about 6 Gy. Immediately after irradiation, primates are treated by oral gavage with lactoferrin at a dose of 1.5 mg/m² or with a vehicle placebo. The primates are dosed once a day for 30 days after exposure. The effect of treatment with oral lactoferrin on increasing the survival of the animals is evaluated. Survival rates are improved in lactoferrin treated animals relative to control animals.

### Example 14

### Dose-Dependent Protection by Oral Lactoferrin Against Radiation-Induced Damage in Non-Human Primates (NHPS)

Non-human primates (n=6/group) are exposed to a whole-body non-lethal dose of ionizing radiation of about 5 Gy. Immediately after irradiation, primates are treated by oral gavage with lactoferrin at doses of 0 (vehicle), 0.36, 0.7 and 1.5 mg/m². The doses are administered to each primate once a day for 30 days after exposure. The blood is collected from primates at various time intervals and the cellular composition of primates' blood is analyzed. The effect of lactoferrin on the mortality of NHPs due to exposure to ionizing radiation, on their blood composition, and their overall health status are evaluated. Mortality, blood cell recovery and/or health status are improved in lactoferrin treated animals relative to control animals.

### REFERENCES CITED

All patents and publications mentioned in the specifications are indicative of the levels of those skilled in the art to which the invention pertains. All patents and publications are herein incorporated by reference to the same extent as if each individual publication was specifically and individually indicated to be incorporated by reference.
Artym J, Zimecki M, Kruzel M L: Reconstitution of the cellular immune response by lactoferrin in cyclophosphamide-treated mice is correlated with renewal of T-cell compartment. Immunobiology, 2003; 207: 187-205.
Bagby G C Jr, McCall E, Layman D L: Regulation of colony-stimulating activity production. Interactions of fibroblasts, mononuclear phagocytes, and lactoferrin. J Clin Invest, 1983; 71: 340-44
Debbabi H, Dubarry M, Rautureau M, Tome D: Bovine lactoferrin induces both mucosal and systemic immune response in mice. J Dairy Res, 1998; 65: 283-93
Ellison R T III, Giehl T J, LaForce F M: Damage of the outer membrane of enteric gram-negative bacteria by lactoferrin and transferrin. Infect Immun, 1988; 56: 2774-81
Hashizume S, Kuroda K, Murakami H: Identification of lactoferrin as an essential growth factor for human lymphocytic cell lines in serum-free medium. Biochim Biophys Acta, 1983; 763: 377-82
Kyte J, Doolittle R F. A simple method for displaying the hydropathic character of a protein. J Mol Biol. 1982 May 5; 157(1):105-32.
Lonnerdal B, Iyer S: Lactoferrin: molecular structure and biological function. Annu Rev Nutr, 1995; 15: 93-110
Mikogami T., Heyman M., Spik G. and Desjeux J. F. (1994) Apical-to-basolateral transepithelial transport of human lactoferrin in the intestinal cell line HT-29cl.19A. Am. J. Physiol. 267: G308-315
Morton D B. Refinement of in vivo tests. Dev Biol Stand. 1999; 101:187-93
Rado T A, Bollekens J, St Laurent G et al: Lactoferrin biosynthesis during granulocytopoiesis. Blood, 1984; 64: 1103-9
Rich I N, Sawatzki G: Lactoferrin, the signal for colony stimulating factor production? Negative-feedback regulation versus supply and-demand regulation of myelopoiesis. Blood Cells, 1989; 15:400-6
Rich I N: The macrophage as a production site for hematopoietic regulator molecules: sensing and responding to normal and pathophysiological signals. Anticancer Res, 1988; 8: 1015-40
Wakabayashi H, Abe S, Okutomi T et al: Cooperative anti-Candida effects of lactoferrin or its peptides in combination with azole antifungal agents. Microbiol Immunol, 1996; 40: 821-25
Zimecki M, Mazurier J, Machnicki M et al: Immunostimulatory activity of lactotransferrin and maturation of CD4− CD8− murine thymocytes. Immunol Lett, 1991; 30: 119-23
Zimecki M, Mazurier J, Spik G, Kapp J A: Human lactoferrin induces phenotypic and functional changes in murine splenic B cells. Immunology, 1995; 86: 122-27
Zimecki M, Spiegel K, Wlaszczyk A et al: Lactoferrin increases the output of neutrophil precursors and attenuates the spontaneous production of TNF-alpha and IL-6 by peripheral blood cells. Arch Immunol Ther Exp (Warsz), 1999; 47: 113-18
Nuijens et al. (U.S. Pat. No. 6,333,311)

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A method of treating a subject exposed to irradiation comprising the step of administering to the subject an effective amount of a lactoferrin composition, wherein said lactoferrin composition decreases morbidity and/or mortality of the subject exposed to irradiation.

2. The method of claim 1 when said lactoferrin composition is administered prior to exposure to irradiation.

3. The method of claim 1 when said lactoferrin composition is administered after the exposure to irradiation.

4. The method of claim 1, wherein said lactoferrin composition is dispersed in a pharmaceutically acceptable carrier.

5. The method of claim 1, wherein the amount of the lactoferrin composition that is administered is about 0.01 to 2.0 g/kg per day.

6. The method of claim 1, wherein the amount of the lactoferrin composition that is administered is from 0.01 to 0.5 g/kg.

7. The method of claim 1, wherein the lactoferrin composition is administered orally or intravenously.

8. The method of claim 7, wherein the said lactoferrin composition is administered as a liquid formulation.

9. The method of claim 7, wherein the said lactoferrin composition is administered as a solid formulation.

10. The method of claim 9, wherein the said solid formulation comprises an enteric coating.

11. The method of claim 1, wherein the lactoferrin composition is administered topically.

12. The method of claim 1, wherein the irradiation is selected from ²³⁵U, ¹³¹I, ¹²³I, ⁹⁹Tc, ²⁰¹Th, ¹³³Xe, ¹²⁵I, ⁶⁰Co, and ¹³⁷Cs, ⁶⁰Co, ¹³⁷Cs, ¹⁹²Ir, ³²P, ⁹⁰Sr, ²²⁶Ra and a combination thereof.

13. A method of treating the sequelae caused by exposure to a dose of ionizing radiation comprising the step of supplementing the mucosal immune system in a subject by orally administering an effective amount of a lactoferrin composition.

14. A method of enhancing a mucosal immune response in the gastrointestinal tract in a subject that received an absorbed dose of ionizing radiation comprising the step of orally administering an effective amount of a lactoferrin composition.

15. The method of claim 14, wherein the lactoferrin composition stimulates the production of a cytokine or a chemokine.

16. The method of claim 14, wherein the lactoferrin composition results in an inhibition of a cytokine or a chemokine.

17. The method of claim 15, wherein the cytokine is selected from the group consisting of interleukin-18 (IL-18), interleukin-12 (IL-12), granulocyte/macrophage colony-stimulating factor (GM-CSF), and gamma interferon (IFN-γ).

18. The method of claim 15, wherein the chemokine is macrophage inflammatory protein 3 alpha (MIP-3α), macrophage inflammatory protein 1 alpha (MIP-1α), macrophage inflammatory protein 1 beta (MIP-1β).

19. The method of claim 16, wherein the cytokine is selected from the group consisting of interleukin-2 (IL-2), interleukin-4 (IL-4), interleukin-5 (IL-5), interleukin-10 (IL-10), and tumor necrosis factor alpha (TNF-α).

20. The method of claim 33, wherein the lactoferrin composition inhibits the production of matrix metalloproteinases (MMPs).

21. The method of claim 17, wherein interleukin-18 or granulocyte/macrophage colony-stimulating factor stimulates the production or activity of immune cells.

22. The method of claim 21, wherein the immune cells are selected from the group consisting of T lymphocytes, natural killer cells, macrophages, dendritic cells, and polymorphonuclear cells.

23. The method of claim 22, wherein the polymorphonuclear cells are neutrophils.

24. The method of claim 22, wherein the T lymphocytes are selected from the group consisting of CD4+, CD8+ and CD3+ T cells.

25. A method of decreasing mortality of a subject that received an absorbed dose of ionizing radiation comprising the step of orally administering to said subject an effective amount of a lactoferrin composition to attenuate the effect of said absorbed dose.

26. A method of attenuating the damaging effects of an absorbed dose of irradiation in a subject comprising the step of orally administering to said subject an effective amount of a lactoferrin composition to attenuate the damaging effect of said absorbed dose.

27. The method of claim 26, wherein attenuating the damage results in a decrease in morbidity of said subjects.

28. The method of claim 26, wherein attenuating the damage results in a decrease in gut-associated systemic bacterial, viral or fungal infections.

29. The method of claim 26, wherein attenuating the damage results in a decrease in mortality of said subjects.

30. A method of attenuating the damaging effects of an absorbed dose of irradiation in a subject comprising the step of orally administering to said subject an effective amount of a lactoferrin composition in combination with a radioprotective agent to attenuate the damaging effect of said absorbed dose.

31. The method of claim 30, wherein the radioprotective agent is granulocyte-stimulating factor (G-CSF) (Filgrastim/(Neupogen)) or Amifostine.

32. A method of treating the sequelae caused by exposure to a dose of ionizing radiation comprising the step of supplementing the mucosal immune system in a subject by topically administering an effective amount of a lactoferrin composition.

33. A method of enhancing an immune response in the dermal tissues in a subject that received an absorbed dose of ionizing radiation resulting in radiation dermatitis comprising the step of topically administering an effective amount of a lactoferrin composition.

34. The method of claim 33, wherein the lactoferrin composition stimulates the production of a cytokine or a chemokine.

35. The method of claim 33, wherein the lactoferrin composition results in an inhibition of a cytokine or a chemokine.

36. The method of claim 35, wherein the cytokine is selected from the group consisting of interleukin-18 (IL-18), interleukin-12 (IL-12), granulocyte/macrophage colony-stimulating factor (GM-CSF), and gamma interferon (IFN-γ).

37. The method of claim 35, wherein the chemokine is macrophage inflammatory protein 3 alpha (MIP-3α), macrophage inflammatory protein 1 alpha (MIP-1α), macrophage inflammatory protein 1 beta (MIP-1β).

38. The method of claim 35, wherein the cytokine is selected from the group consisting of interleukin-2 (IL-2), interleukin-4 (IL-4), interleukin-5 (IL-5), interleukin-10 (IL-10), and tumor necrosis factor alpha (TNF-α).

39. The method of claim 33, wherein the lactoferrin composition inhibits the production of matrix metalloproteinases (MMPs).

40. The method of claim 36, wherein interleukin-18 or granulocyte/macrophage colony-stimulating factor stimulates the production or activity of immune cells.

41. The method of claim 40, wherein the immune cells are selected from the group consisting of T lymphocytes, natural killer cells, macrophages, dendritic cells, and polymorphonuclear cells.

42. The method of claim 41, wherein the polymorphonuclear cells are neutrophils.

43. The method of claim 41, wherein the T lymphocytes are selected from the group consisting of CD4+, CD8+ and CD3+ T cells.
